(19) United States
(12) Patent Application Publication
(10) Pub. No.: US 2021/0396662 A1
(43) Pub. Date: Dec. 23, 2021

(54) METHODS AND SYSTEMS FOR PREDICTING OPTICAL PROPERTIES OF A SAMPLE USING DIFFUSE REFLECTANCE SPECTROSCOPY

(71) Applicant: SAMSUNG ELECTRONICS CO., LTD., Suwon-si (KR)

(72) Inventors: Ibrahim A, Bangalore (IN); Sreejith Kallummil, Bangalore (IN); Sujit Jos, Bangalore (IN); Karam Choi, Suwon-si (KR); Sang Kyu Kim, Suwon-si (KR)

(73) Assignee: SAMSUNG ELECTRONICS CO., LTD., Suwon-si (KR)

(21) Appl. No.: 17/348,278

(22) Filed: Jun. 15, 2021

(30) Foreign Application Priority Data
Jun. 19, 2020 (IN) 202041025972
May 11, 2021 (KR) 10-2021-0060960

(51) Int. Cl. G01N 21/47; G06N 3/04; G01J 3/02

(52) U.S. Cl. CPC G01N 21/4738; G01J 3/021; G06N 3/04

(57) ABSTRACT

Provided is a method for predicting optical properties of a sample, the method including obtaining, by a device, a plurality of diffuse reflectance values based on optical energy diffusely reflected from the sample, generating, by a multi-layered Deep Fully Connected Neural Network (DFCNN) in the device, a first set of intermediate values by non-linearly mapping the plurality of diffuse reflectance values to the first set of intermediate values, generating, by a One-Dimensional-Convolutional Neural Network (1D-CNN) in the device, a second set of intermediate values by non-linearly mapping the plurality of diffuse reflectance values to the second set of intermediate values, and predicting, by the device, values of the optical properties of the sample based on the first set of intermediate values and the second set of intermediate values.

### CROSS-REFERENCE TO RELATED APPLICATIONS

This application claims priority to Korean Patent Application No. 10-2021-0060960, filed on May 11, 2021 in the Korean Intellectual Property Office, and Indian Patent Application No. 202041025972, filed on Jun. 19, 2020, in the Intellectual Property India, the disclosures of which are incorporated by reference herein in their entireties.

### BACKGROUND

### 1. Field

Embodiments herein relate to Diffuse Reflectance Spectroscopy (DRS), and more particularly to methods and systems for predicting optical properties of a sample using DRS.

### 2. Description of Related Art

Optical energy comprising of photons may be projected on a sample, wherein a portion of the projected optical energy may be diffusely reflected from within the sample, hereby creating a diffuse reflectance spectrum. Spectroscopic features of the diffuse reflectance spectrum may be used for determining optical properties of the sample. The diffuse reflectance spectrum may be compared with a reference diffuse reflectance spectrum, in order to determine the optical properties of the sample. In an example, when the sample is a skin, the characteristics of the skin may be predicted using the diffuse reflectance spectrum, obtained based on optical energy reflected from different regions of the skin.

Currently, optical properties of a sample, based on diffuse reflectance spectrum, are predicted using iterative inverse models (such as Monte Carlo, lookup tables, Feedforward Artificial Neural Network (FANN), and so on) and/or Inverse Artificial Neural Network (IANN). The reference value of an optical property of the sample may vary. There may be a range, within which the reference value of the optical property may vary. The predicted value of the optical property of the sample may be compared against the reference value of the optical property, for computing an error in the prediction. The error may be minimized using an iterative cost function, which may improve the accuracy of the predicted values of the optical property.

The cost functions used in these models, however, are based on mean squared error criterion. Therefore, if the range of reference value of an optical property is smaller and if the difference between the predicted value of the optical property and the reference value of the optical property is smaller, the mean squared error will be small and will likely be ignored. Consequently, the error in the predicted value will not be detected. The models will consider that the predicted values are correct and will not be trained further. As the predicted values of the optical properties are not tuned sufficiently by the models, the prediction accuracy may be affected.

### SUMMARY

One or more example embodiments provide methods and systems for predicting optical properties of a sample using Diffuse Reflectance Spectroscopy (DRS).

One or more example embodiments also provide a Hybrid Deep Neural Network (HDNN) architecture for predicting the optical properties of the sample, wherein the HDNN architecture includes a Deep Fully Connected Neural Network (DFCNN) in parallel with a One-Dimensional-Convolutional Neural Network (1D-CNN), a merging neural network, and an output neural network, wherein the DFCNN, the 1D-CNN, the merging neural network, and the output neural network, map the DRS to a plurality of output values corresponding to predicted values of the optical properties.

One or more example embodiments also provide a mean square weighted error cost function to minimize errors between the predicted values of the optical properties and reference values of the optical properties, wherein the cost function includes a weight factor, which is assigned to the optical properties based on the ranges of the reference values of the optical properties, wherein the ranges indicate the differences between maximum reference values and minimum reference values of the optical properties.

According to an aspect of an example embodiment, there is provided a method for predicting optical properties of a sample, the method including obtaining, by a device, a plurality of diffuse reflectance values based on optical energy diffusely reflected from the sample, generating, by a multi-layered Deep Fully Connected Neural Network (DFCNN) in the device, a first set of intermediate values by non-linearly mapping the plurality of diffuse reflectance values to the first set of intermediate values, generating, by a One-Dimensional-Convolutional Neural Network (1D-CNN) in the device, a second set of intermediate values by non-linearly mapping the plurality of diffuse reflectance values to the second set of intermediate values, and predicting, by the device, values of the optical properties of the sample based on the first set of intermediate values and the second set of intermediate values.

The plurality of diffuse reflectance values may be provided as an input feature vector to the DFCNN, and the plurality of diffuse reflectance values may correspond to features of the input feature vector.

The plurality of diffuse reflectance values may be provided as an input tensor to the 1D-CNN, and the 1D-CNN may obtain shape characteristics of the plurality of diffuse reflectance values.

The optical properties are predicted by generating a merged set of intermediate layer output values by merging the first set of intermediate values and the second set of intermediate values by a merging neural network, and reducing the merged set of intermediate values to a predefined number of output values, wherein the intermediate values in the merged set is non-linearly mapped to the predefined number of output values by an output neural network including at least one layer.

The DFCNN, the 1D-CNN, the merging neural network, and the output neural network may be trained to predict the values of the optical properties based on a mean square weighted error cost function.

A value of the mean square weighted error cost function may be determined based on an error vector and a weight vector, wherein the error vector is a difference between a first vector, corresponding to the values of the optical properties predicted during the training, and a second vector, corresponding to specified reference values of the optical properties, and wherein the weight vector corresponds to weight factors assigned to the optical properties.

Magnitudes of dimensions of the weight vector may be inversely proportional to ranges of the specified reference values of the optical properties, and the ranges of the specified reference values of the optical properties may correspond to differences between maximum specified reference values of the optical properties and minimum specified reference values of the optical properties.

The specified reference values of the optical properties may correspond to reference values of diffuse reflectance, and the reference values of diffuse reflectance may be provided as input to the DFCNN, the 1D-CNN, the merging neural network and the output neural network, during the training.

According to another aspect of an example embodiment, there is provided a device configured to predict optical properties of a sample, the device including at least one processor configured to obtain a plurality of diffuse reflectance values based on optical energy diffusely reflected from within the sample, generate, by a multi-layered Deep Fully Connected Neural Network (DFCNN) in the device, a first set of intermediate values by non-linearly mapping the plurality of diffuse reflectance values to the first set of intermediate values, generate, by a One-Dimensional-Convolutional Neural Network (1D-CNN) in the device, a second set of intermediate values by non-linearly mapping the plurality of diffuse reflectance values to the second set of intermediate values, and predict values of the optical properties of the sample based on the first set of intermediate values and the second set of intermediate values.

The plurality of diffuse reflectance values may be provided as an input feature vector to the DFCNN, and the plurality of diffuse reflectance values may correspond to features of the input feature vector.

The plurality of diffuse reflectance values may be provided as an input tensor to the 1D-CNN, and the 1D-CNN may obtain shape characteristics of the plurality of diffuse reflectance values.

The at least one processor is further configured to predict the optical properties by generating a merged set of intermediate layer output values by merging the first set of intermediate values and the second set of intermediate values by a merging neural network, and reducing the merged set of intermediate values to a predefined number of output values, wherein the intermediate values in the merged set is non-linearly mapped to a predefined number of output values by an output neural network including of at least one layer.

The DFCNN, the 1D-CNN, the merging neural network, and the output neural network, may be configured to be trained to predict the values of the optical properties based on a mean square weighted error cost function.

A value of the mean square weighted error cost function may be determined based on an error vector and a weight vector, wherein the error vector is a difference between a first vector, corresponding to the values of the optical properties predicted during the training, and a second vector, corresponding to specified reference values of the optical properties, and wherein the weight vector corresponds to weight factors assigned to the optical properties.

Magnitudes of dimensions of the weight vector may be inversely proportional to ranges of the specified reference values of the optical properties, and wherein the ranges of the specified reference values of the optical properties correspond to differences between maximum specified reference values of the optical properties and minimum specified reference values of the optical properties.

The specified reference values of the optical properties may correspond with reference values of diffuse reflectance, and the reference values of diffuse reflectance may be provided as input to the DFCNN, the 1D-CNN, the merging neural network and the output neural network, during the training.

### BRIEF DESCRIPTION OF THE DRAWINGS

The above and/or other aspects, features, and advantages of example embodiments will be more apparent from the following description taken in conjunction with the accompanying drawings, in which:

FIG. 1 illustrates various units of a device configured to predict the optical properties of a sample using Diffuse Reflectance Spectroscopy (DRS) according to example embodiments;

FIGS. 2A and 2B illustrate the architecture of the processor of the device, configured to predict the optical properties of the sample, according to example embodiments; and

FIG. 3 is a flowchart illustrating a method for predicting the optical properties of the sample using DRS according to example embodiments.

### DETAILED DESCRIPTION

The example embodiments herein and the various features and advantageous details thereof are explained more fully with reference to the non-limiting example embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the example embodiments herein may be practiced and to further enable those of skill in the art to practice the embodiments herein. Accordingly, the examples should not be construed as limiting the scope of the embodiments herein.

Example embodiments herein disclose methods and systems for predicting optical properties of a sample. The optical properties are predicted using Diffuse Reflectance Spectroscopy (DRS). The example embodiments include obtaining a plurality of diffuse reflectance values based on optical energy, obtained from detectors kept at increasing source detector separations, which is diffusely reflected from within the sample. The example embodiments include providing the diffuse reflectance values to a neural network architecture, which includes a fully connected sub-network and a convolutional sub-network. The architecture predicts the optical properties of the sample based on the diffuse reflectance of the sample.

The example embodiments include providing an N-dimensional feature vector to the fully connected sub-network, and providing a (N, 1) tensor, to the convolutional sub-network, where N specifies the number of diffuse reflectance values corresponding to N source detector separations. The feature vector and the tensor specify the different means of interpretation, of the diffuse reflectance values, by the fully connected sub-network and the convolutional sub-network respectively. The fully connected sub-network may include of a plurality of neural network layers, where a non-linear mapping is performed at each layer to generate output values from input values. The feature vector provided as input to the first layer of the fully connected sub-network and the (N, 1) tensor provided to the convolutional sub-network is mapped to optical properties. In an example embodiment, the Convolutional sub-network is a One Dimensional Convolutional Neural Network (1D-CNN).

The 1D-CNN captures the shape characteristics of the diffuse reflectance values. The shape characteristics may be mapped to the optical properties of the sample. Intermediate features provided by the fully connected sub-network and the 1D-CNN may be merged using a merging neural network, which is then reduced by an output neural network to provide the predicted values of the optical properties of the sample.

The example embodiments include training the neural network architecture for predicting values of the optical properties. The example embodiments include determining reference values of diffuse reflectance by specifying reference values of the optical properties, which will be used to train the neural network architecture. The predicted values of the optical properties may be compared with the reference values of the optical properties. The example embodiments include minimizing errors between the predicted reference values of the optical properties and the reference values of the optical properties by appropriately tuning the neural network using back propagation.

The example embodiments include providing a mean square weighted error cost function to minimize errors between the predicted values of the optical properties and reference values of the optical properties. Training the neural network using this cost function, allows improving the accuracy of predicted values of the optical properties. The cost function includes a weight factor, which is assigned to the optical properties based on the ranges of the reference values of the optical properties. The ranges define the differences between maximum reference values and minimum reference values of the respective optical properties. The weight factor allows more accurate prediction of the optical properties of the sample, irrespective of the ranges of the reference values of the optical properties.

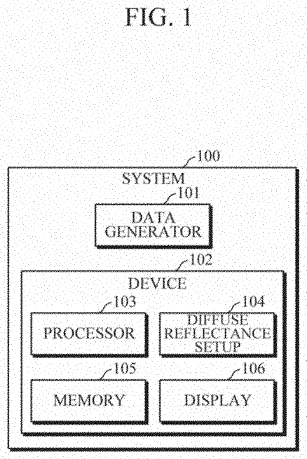

FIG. 1 illustrates various units of a system 100 configured to predict the optical properties of a sample using DRS according to example embodiments. As illustrated in FIG. 1, the system 100 may include a data generator 101 and a device 102. The device 102 may include a processor 103, a diffuse reflectance setup 104, a memory 105, and a display 106.

The data generator 101 may be used for obtaining data comprising of reference values of diffuse reflectance based on specified range of reference values of optical properties of a sample. The reference values of the optical properties of the sample may be specified based on standard values of parameters such as absorption coefficient, scattering coefficient, and on the like, which defines the characteristics of the sample.

The processor 103 may be trained to predict the optical properties of sample based on the reference values of diffuse reflectance. During the training phase, the reference values of diffuse reflectance are provided as an input to the processor 103. Based on the reference values of diffuse reflectance, the processor 103 predicts values of optical properties.

In an example embodiment, the processor 103 may determine a relative error based on the difference between the predicted values of the optical properties and the specified reference values of the optical properties. In an example embodiment, the relative error may be a K-dimensional vector, wherein K may specify the number of optical properties predicted by the system 100. The processor 103 uses a mean square weighted error cost function to minimize the relative error and improve the accuracy of the predicted values of the optical properties.

The cost function includes a weight factor. The weights may be assigned to different optical properties based on the ranges of the reference values of the optical properties. In an example embodiment, the weight factor is reciprocal to the range of the reference value of the optical property. The ranges may indicate the differences between maximum reference values and minimum reference values of the optical properties. The weight factor is a K-dimensional vector, where K may specify the number of optical properties predicted by the system 100. The magnitude of each dimension of the weight factor depends on the difference between the maximum reference value and minimum reference value of a particular optical property.

For example, the range of the reference value of a first optical property may be 16, where the minimum reference value is 15 and the maximum reference value is 31. The range of the reference value of a second optical property may be 0.08, where the minimum reference value is 0.74 and the maximum reference value is 0.82. In this example, the magnitude of the first dimension of the weight factor, which the processor 103 assigns, is likely to be smaller than that compared to the magnitude of the second dimension of the weight factor corresponding to the second optical property, as the range of the reference value of the second optical property (0.08) is less than the range of the reference value of the first optical property (16).

Consider that y_{ref}ⁱ is a K-dimensional vector representing the reference value of the optical properties, y_{pred}ⁱ is a K-dimensional vector representing the predicted value of the optical properties, and weights_{OP} is the K-dimensional weight vector. As y_{ref}ⁱ and y_{pred}ⁱ are K-dimensional vectors, (y_{ref}ⁱ−y_{pred}ⁱ) is also a K-dimensional vector representing the relative error between the predicted and reference values of the optical properties. The cost function may be represented as Equation 1 shown below.

$$\frac{1}{N} {\sum }_{i = 1}^{N} {‖ ( {y}_{ref}^{i} - {y}_{pred}^{i} ) * w e i g h t {s}_{O P} ‖}_{2}^{2} \qquad [ Equation 1 ]$$

Here, * denotes the dot product over vectors and i denotes the i th training sample and N denotes the total number of training samples.

The mean square weighted error cost function considers the range of reference values of the optical properties to determine the value of the cost function, as the magnitudes assigned to different dimensions of the weight vector are based on the specified ranges of the reference values of the respective optical properties. The optical property specified with a small reference value range is higher compared to an optical property specified with a greater reference value range. This may improve the accuracy of prediction. For example, when the second optical property is predicted to be 0.75, and the reference value of the second optical property is 0.81, the difference between the predicted value and the reference value (relative error) is 0.06. When the first optical property is predicted to be 29, and the reference value is 21, the difference between the predicted value and the reference value (relative error) is 8.

If the magnitudes assigned to different dimensions of the weight factor are not considered, the difference (relative error) will be considered for determining the value of the cost function. The contribution of the relative error corresponding to the second optical property, to the cost function, would be negligible compared to that of the first optical property, the differences being 0.06 and 8, respectively. As the contribution of the relative error to the cost function, corresponding to the second optical property, is smaller, the predicted value of the second optical property would be considered more accurate. Thus, the processor 103 will not be trained to more accurately predict the value of the second optical property.

The processor 103 will be trained to more accurately predict the value of the first optical property, as the value of the cost function for the first optical property is significantly higher. Thus, the ranges of the reference values of the optical properties may affect the accuracy in predicting the actual values of the optical properties.

The magnitude of the dimension of the weight factor corresponding to the second optical property will be 12.5 reciprocal to the range of the reference value of the optical property, i.e., 0.08. The magnitude of the dimension of the weight factor corresponding to the first optical property will be 0.0625 reciprocal to the range of the reference value of the optical property, i.e., 16. The product of the magnitude of the dimension of the weight factor and the difference (relative error), for the second optical property, will be 0.75 (12.5*0.06). The product of the magnitude of the dimension of the weight factor and the difference (relative error), for the first optical property, will be 0.5 (0.0625*8).

The product of the weight factor and the difference is included in the cost function, thereby balancing the smaller range of the reference value with a larger value of weight factor. Thus, the processor 103 considers the difference between the predicted value and reference values of the first and second optical properties equally, despite significant variations between the ranges of the reference values of the first and second optical properties. This helps in tuning the parameters to have accurate predictions evenly for all the optical properties. This increases the accuracy of the predicted values of all the optical properties of the sample. After training, processor 103 may be used for predicting optical properties of the sample after using diffuse reflectance values, obtained using different source detector separations.

The diffuse reflectance setup 104 includes a source fiber and a detector fiber. The source fiber and the detector fiber may each include at least one probe. The source fiber and the detector fiber may be spatially separated. Optical energy, comprising of photons may be projected on the sample through the source fiber probe. In an example embodiment, the optical energy may be visible light. In an example embodiment, the optical energy may be near-infrared (NIR) light. Some of the photons may be absorbed by the sample, while some of the photons may be reflected either in specular manner or diffusely.

The photons, which are diffusely reflected from the sample, may be collected using the detector fiber probes, which may have multiple source detector separations. In an example, consider that there are nine source detector separations. The source detector separations may be placed equidistant from each other. The optical properties of the sample may be predicted based on a path followed by the photons, which are diffusely reflected from within the sample. In an example embodiment, diffuse reflectance values may be obtained based on the reflected optical energy, collected through the detector fiber probes at different source detector separations. In this example, nine diffuse reflectance values may be obtained. The optical properties of the sample may be predicted based on these nine diffuse reflectance values.

Example embodiments have been explained by considering the skin of a human as an example of a sample. The system 100 may predict the optical properties of the skin based on the diffuse reflectance values. Further, the characteristics of the skin may be predicted based on the optical properties of the skin. The system 100 may consider the three layers of the skin, for example, dermis, epidermis, and subcutis. The system 100 may predict six optical properties of the skin, for example, absorptivity of the dermis, scattering from with the dermis, thickness of the dermis, absorptivity of the epidermis, thickness of the epidermis, and the absorptivity of the subcutis. Therefore, the system 100 may generate six values as an output.

In an example embodiment, the processor 103 may be a neural network processor. The processor 103 may have Hybrid Deep Neural Network (HDNN) architecture. The HDNN may include a Deep Fully Connected Neural Network (DFCNN), a One-Dimensional-Convolutional Neural Network (1D-CNN) a merging neural network, and an output neural network. The DFCNN and the 1D-CNN may operate in parallel. The DFCNN and the 1D-CNN may receive the diffuse reflectance values from the source detector separations as inputs. The DFCNN considers the diffuse reflectance values as a feature vector and the 1D-CNN considers the diffuse reflectance values as a tensor. The diffuse reflectance values may be obtained when the optical energy is diffusely reflected from within the sample. If the sample is a human skin, the diffuse reflectance values are obtained when optical energy projected on the surface of the skin is diffusely reflected from different levels of the skin.

The processor 103 may obtain nine diffuse reflectance values from the nine source detector separations. The value of the diffuse reflectance obtained from the source detector separation placed closest to a focal point on which the optical energy is projected may be the highest. The values of diffuse reflectance obtained from the different source detector separations may be related to each other. In an example embodiment, the values of diffuse reflectance may decay exponentially with respect to the distance between the source detector separation and the focal point. The processor 103 may determine the decaying exponential function.

The diffuse reflectance values obtained from the nine source detector separations may be fed to the DFCNN and the 1D-CNN. The DFCNN considers the nine diffuse reflectance values (fed as input) as a 9D feature vector. In an example embodiment, the DFCNN may be a 7-layered fully connected neural network. The DFCNN may map the input diffuse reflectance values non-linearly to a plurality of output values, which may correspond to the optical properties of the skin. In an example embodiment, the DFCNN may generate a 50 intermediate values, that may be further mapped to the optical properties of the skin, based on the 9D input vector representing the diffuse reflectance values through the seven layers of the DFCNN.

The 1D-CNN considers the nine diffuse reflectance values (fed as input) as a (9, 1) tensor. The 1D-CNN may capture the shape characteristics of the diffuse reflectance values and map the shape characteristics to the optical properties of the skin. In an example embodiment, the kernel size of the 1D-CNN is 3 and the number of filters used is 8. The (9, 1) tensor may be convoluted with the kernel to generate a (9, 8) tensor. The output of the convolution is further convoluted to generate a (5, 8) tensor. In an example embodiment, the 1D-CNN may generate 40 intermediate values, that may be further mapped to the optical properties of the skin, based on the (9, 1) input tensor representing the diffuse reflectance values.

The processor 103 may merge the outputs of the DFCNN and the 1D-CNN using a merging neural network. Considering the example, the 50 intermediate values (from the DFCNN) may be merged with the 40 intermediate values (from the 1D-CNN) to generate a 90 intermediate values. The 90 intermediate values may be reduced to a 6-valued output representing the predicted values of the optical properties, for example, the absorptivity of the dermis, scattering from with the dermis, thickness of the dermis, absorptivity of the epidermis, thickness of the epidermis, and the absorptivity of the subcutis.

The processor 103 may reduce the outputs of the merging neural network using the output neural network. In an example embodiment, the processor 103 reduces the 90 intermediate values to the 6-valued output using two layers of the output neural network. In the first layer, the processor 103 reduces the 90 intermediate values to 15 intermediate values. In the second layer, the processor 103 reduces the 15 intermediate values to 6 output values, corresponding to the predicted values of the optical properties.

FIG. 1 shows exemplary units of the system 100, but embodiments are not limited thereto. In other example embodiments, the system 100 may include less or more number of units. Further, the labels or names of the units are used only for illustrative purpose and does not limit the scope. One or more units may be combined together to perform same or substantially similar function in the system 100.

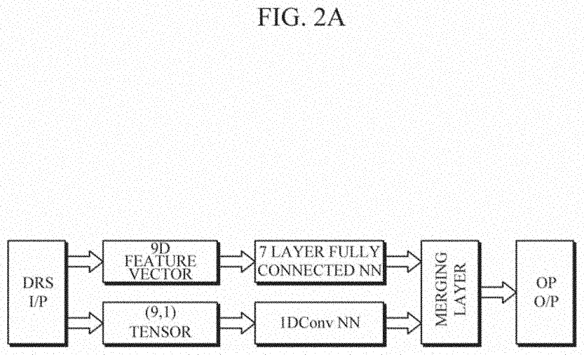

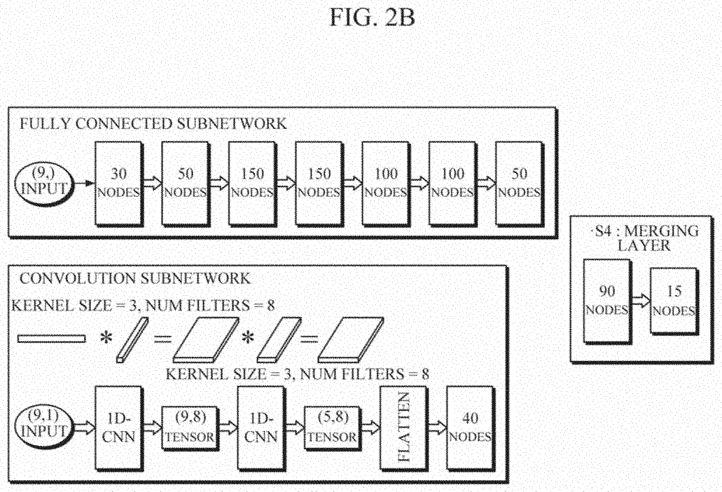

FIGS. 2A and 2B illustrate the architecture of the processor 103 of the system 100, configured to predict the optical properties of the sample, according to example embodiments. As depicted in FIG. 2A, the input to the processor 103 is a 9-valued diffuse reflectance. Considering that the source detector separations are equidistant from each other, the diffusely reflected optical energy is collected from the source detector separations. The diffuse reflectance values are obtained based on the diffusely reflected optical energy.

The diffuse reflectance values may be normalized such that the values of the diffuse reflectance are between 0 and 1. As the diffuse reflectance values are dependent on each other, each diffuse reflectance value may be normalized using the highest diffuse reflectance value. Hence, after normalization, the highest value of diffuse reflectance will be 1 and the rest of the diffuse reflectance values will be less than 1.

The processor 103 has an HDNN architecture, which includes of the DFCNN, 1D-CNN, merging neural network, and output neural network. The DFCNN and 1D-CNN may operate in parallel. The DFCNN considers the normalized diffuse reflectance values as a feature vector. As there are nine normalized values, the input to the DFCNN is a 9D feature vector.

The 1D-CNN considers the normalized diffuse reflectance values as a (9, 1) tensor. The 1D-CNN determines the relationship between the diffuse reflectance values obtained from the different source detector separations. The shape defining the variation of the diffuse reflectance values may be derived based on the relationship between the diffuse reflectance values. The value of the diffuse reflectance obtained from the source detector separation placed closest to the focal point is the highest. The values of diffuse reflectance obtained from the other source detector separations decrease with respect to the distance between the source detector separation and the focal point. The 1D-CNN may determine the exponentially decaying shape defining the variation of the diffuse reflectance values.

As depicted in the example in FIG. 2B, the DFCNN is a 7-layered fully connected network. Each layer is a neural network comprising of nodes, wherein the number of input nodes and the number of output nodes may be same or different. The DFCNN may non-linearly map the nine diffuse reflectance values with the optical properties of the sample. The first layer includes 9 input nodes and the 9 features (diffuse reflectance values) are fed to the input nodes. The first layer includes of 30 output nodes. Therefore, the 9 input features are mapped to 30 output features. Similarly, in the second layer, the 30 features are mapped to 50 features. In the seventh layer, there are 100 input nodes and 50 output nodes. The 100 features, obtained from the sixth layer are mapped to 50 features. The DFCNN, thus, generates 50 values as intermediate layer output, which may be mapped to the optical properties of the sample.

The 1D-CNN considers the nine diffuse reflectance values (fed as input) as a (9, 1) tensor. The 1D-CNN is having 8 filters and the kernel size is 3. The (9, 1) input tensor may be convoluted with the kernel to generate a (9, 8) tensor. The (9, 8) tensor is further convoluted with the kernel to generate a (5, 8) tensor. The (5, 8) tensor is the output of the 1D-CNN, which may be flattened to generate 40 values as output. The 1D-CNN may capture the shape characteristics of the diffuse reflectance values, in the (9, 1) tensor form, using the convolution procedure. The shape characteristics are represented in the 40 intermediate layer output values.

The 50 values generated by the DFCNN and the 40 values generated by the 1D-CNN may be merged using the merging neural network to generate 90 intermediate output values. The 90 values may be reduced to 6 values which are the predicted values of the optical properties. The reduction from 90 values to 6 values may take place in two layers of the output neural network. In the first layer, the 90 input values are reduced to 15 output values. In the second layer, the 15 input values are reduced to 6 output values.

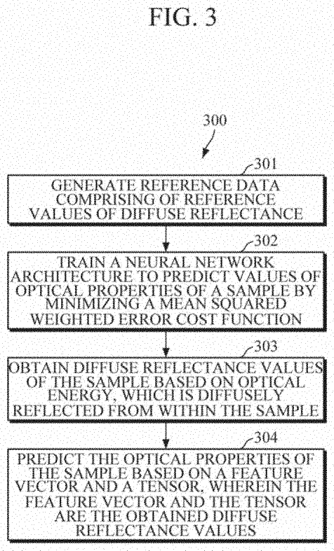

FIG. 3 is a flowchart 300 depicting a method for predicting the optical properties of the sample using DRS according to example embodiments as disclosed herein. At step 301, the method includes generating reference data comprising of reference values of diffuse reflectance. The example embodiments include specifying a range of reference values of the optical properties. The example embodiments include generating the reference values of diffuse reflectance using the specified range of the reference values of the optical properties. For each optical property, the example embodiments include storing the specified range of reference values of the optical properties and the corresponding reference values of diffuse reflectance as reference data.

At step 302, the method includes training the HDNN architecture to predict values of the optical properties of a sample by minimizing a mean squared weighted error cost function. The example embodiments include computing differences between values of the optical properties of the sample, predicted by the HDNN architecture during the training phase, and the reference values of the optical properties. The specified reference values of the optical properties may be compared with the predicted values of the optical properties, for error computation.

The example embodiments include determining relative errors based on the differences between the predicted values and the reference values of the respective optical properties. The mean square weighted error cost function is utilized to train the DFCNN and the 1D-CNN using back propagation, such that the error is minimized and the accuracy of prediction of the optical properties is improved.

The cost function includes a weight factor, where the weight factor is a K-dimensional vector, and the magnitudes of different dimensions of the weight factor is based on the ranges of the reference values of the respective optical properties. The ranges represent the difference between the specified maximum reference values and specified minimum reference values of the respective optical properties.

The example embodiments include assigning a greater magnitude to a dimension of the weight vector corresponding to an optical property, which has a smaller range of reference value. In an example embodiment, the magnitude of the dimension of the weight vector is reciprocal to the range of the reference value of the optical property. If y_{ref}ⁱ is a K-dimensional vector representing the reference value of the optical properties, y_{pred}ⁱ is a K-dimensional vector representing the predicted value of the optical properties, and weights_{OP} is the K-dimensional weight vector. The cost function may be represented as Equation 2 shown below.

$$\frac{1}{N} {\sum }_{i = 1}^{N} {‖ ( {y}_{ref}^{i} - {y}_{pred}^{i} ) * w e i g h t {s}_{O P} ‖}_{2}^{2} \qquad [ Equation 2 ]$$

Here, * denotes the dot product over vectors and i denotes the i th training sample and N denotes the total number of training samples

If the magnitudes assigned to different dimensions of the weight factor are not considered, the value of the cost function will depend on the differences between the predicted values and the reference values of the respective optical properties. If the difference between the predicted value and the reference value, for an optical property, is smaller, and the range of reference value is smaller, and the contribution of the optical property to the cost function would be negligible. The predicted value of the optical property would be considered accurate and the DFCNN and the 1D-CNN would not be trained to more accurately predict the value of the optical property.

The magnitude assigned to the dimension of the weight factor representing the optical property with a small reference value range is greater as compared to the weight factor of an optical property with a large reference value range. As such, the cost function (using the weight vector) considers the ranges of the reference values of the different optical properties, irrespective of the differences between the predicted values and the reference values of the respective optical properties, to determine whether the predicted values are accurate. This may improve the prediction accuracy.

At step 303, the method includes obtaining diffuse reflectance values of a sample based on optical energy, which is diffusely reflected from within the sample. The optical energy, comprising of photons, may be projected on the sample, wherein some of the photons are absorbed by the sample and some of the photons are reflected diffusely.

The diffusely reflected photons may be collected using source detector separations. The source detector separations may be placed equidistant from each other. The optical properties of the sample may be predicted based on reflectance of the photons. In an example embodiment, the diffuse reflectance values are obtained based on the reflected optical energy, collected through the source detector separations. For example, if the sample is a human skin, the diffuse reflectance values may be obtained when optical energy is projected on the surface of the skin. The optical energy, diffusely reflected from different layers of the skin, may be collected.

At step 304, the method includes predicting the optical properties of the sample based on a feature vector and a tensor. The feature vector and the tensor are interpretations of diffuse reflectance values. The feature vector includes of the diffuse reflectance values obtained from the source detector separations, representing the features of and the feature vector. The tensor includes of the diffuse reflectance values in a tensor form. The tensor may be used for capturing the shape characteristics of the diffuse reflectance values, from which the relationship between the diffuse reflectance values may be derived.

The example embodiments utilize the HDNN architecture for predicting the optical properties of the sample, which includes the DFCNN and the 1D-CNN. The DFCNN and the 1D-CNN operate in parallel. The feature vector is provided as input to the DFCNN and the shape vector is provided as input to the 1D-CNN.

The DFCNN considers the diffuse reflectance values as an N-Dimensional feature vector, wherein N is the number of diffuse reflectance values. In an example embodiment, the DFCNN is a fully connected neural network including a plurality of layers. Each layer includes of an input node and an output node, and the number of input nodes and the number of output nodes of the neural network may be the same or different. The DFCNN may non-linearly map the diffuse reflectance values to a first set of intermediate values.

The 1D-CNN considers the diffuse reflectance values as a (N, 1) tensor. The 1D-CNN may capture the shape characteristics of the diffuse reflectance values and map the shape characteristics to the optical properties of the sample. The input tensor may be convolved with a kernel of a predefined size. The 1D-CNN may have a predefined number of filters. The number of output values generated by the 1D-CNN may depend on the predefined number of filters and the number of diffuse reflectance values fed as input to the 1D-CNN in a tensor form. The output values generated by the 1D-CNN may be considered as a second set of intermediate values.

The example embodiments include merging the intermediate values generated by the DFCNN and the 1D-CNN using a merging neural network. For example, the merging neural network generates 90 intermediate outputs, where the first set includes of 50 intermediate values generated by the DFCNN and the second set includes of 40 intermediate values generated by the 1D-CNN. The output generated by the merging neural network may be reduced in a predefined number of stages by an output neural network. For example, the reduction may take place in two stages. In the first stage, the 90 values are reduced to 15 values. In the second stage, the 15 values are reduced to 6 values. The 6 values represent the predicted values of the six optical properties.

It may be noted that steps 301 and 302 are performed in order to train the HDNN architecture for predicting the optical properties of the sample. Once the HDNN architecture has been trained and deployed, further performing the steps 301 and 302 may not be necessary. The example embodiments perform steps 303 and 304 for predicting optical properties of different samples.

The various actions in the flowchart 300 may be performed in the order presented, in a different order, or simultaneously. Further, in some embodiments, some actions listed in FIG. 3 may be omitted.

The example embodiments disclosed herein may be implemented through at least one software program running on at least one hardware device and performing network management functions to control the network elements. The network elements shown in FIGS. 2A and 2B include blocks which may be at least one of a hardware device, or a combination of hardware device and software module.

The example embodiments disclosed herein describe methods and systems for predicting the optical properties of the sample using DRS. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in an example embodiment through or together with a software program written in, for example, very high speed integrated circuit Hardware Description Language (VHDL) another programming language, or implemented by one or more VHDL or several software modules being executed on at least one hardware device. The hardware device may be any kind of portable device that may be programmed. The device may also include means which could be, for example, hardware means such as, for example, an application-specific integrated circuit (ASIC), or a combination of hardware and software means, for example, an ASIC and a field-programmable gate array (FPGA), or at least one microprocessor and at least one memory with software modules located therein. The method according to example embodiments described herein could be implemented partly in hardware and partly in software. Alternatively, the example embodiments may be implemented on different hardware devices, for example, using a plurality of central processing units (CPUs).

It should be understood that example embodiments described herein should be considered in a descriptive sense only and not for purposes of limitation. Descriptions of features or aspects within each example embodiment should typically be considered as available for other similar features or aspects in other embodiments. While example embodiments have been described with reference to the figures, it will be understood by those of ordinary skill in the art that various changes in form and details may be made therein without departing from the spirit and scope as defined by the following claims and their equivalents.

What is claimed is:

1. A method for predicting optical properties of a sample, the method comprising:
obtaining, by a device, a plurality of diffuse reflectance values based on optical energy diffusely reflected from the sample;
generating, by a multi-layered Deep Fully Connected Neural Network (DFCNN) in the device, a first set of intermediate values by non-linearly mapping the plurality of diffuse reflectance values to the first set of intermediate values;
generating, by a One-Dimensional-Convolutional Neural Network (1D-CNN) in the device, a second set of intermediate values by non-linearly mapping the plurality of diffuse reflectance values to the second set of intermediate values; and
predicting, by the device, values of the optical properties of the sample based on the first set of intermediate values and the second set of intermediate values.

2. The method, as claimed in claim 1, wherein the plurality of diffuse reflectance values is provided as an input feature vector to the DFCNN, and
wherein the plurality of diffuse reflectance values correspond to features of the input feature vector.

3. The method, as claimed in claim 1, wherein the plurality of diffuse reflectance values is provided as an input tensor to the 1D-CNN, and
wherein the 1D-CNN obtains shape characteristics of the plurality of diffuse reflectance values.

4. The method, as claimed in claim 1, wherein the optical properties are predicted by:
generating a merged set of intermediate layer output values by merging the first set of intermediate values and the second set of intermediate values by a merging neural network; and
reducing the merged set of intermediate values to a predefined number of output values,
wherein the intermediate values in the merged set is non-linearly mapped to the predefined number of output values by an output neural network comprising at least one layer.

5. The method, as claimed in claim 4, wherein the DFCNN, the 1D-CNN, the merging neural network, and the output neural network are trained to predict the values of the optical properties based on a mean square weighted error cost function.

6. The method, as claimed in claim 5, wherein a value of the mean square weighted error cost function is determined based on an error vector and a weight vector,
wherein the error vector is a difference between a first vector, corresponding to the values of the optical properties predicted during the training, and a second vector, corresponding to specified reference values of the optical properties, and
wherein the weight vector corresponds to weight factors assigned to the optical properties.

7. The method, as claimed in claim 6, wherein magnitudes of dimensions of the weight vector is inversely proportional to ranges of the specified reference values of the optical properties, and
wherein the ranges of the specified reference values of the optical properties correspond to differences between maximum specified reference values of the optical properties and minimum specified reference values of the optical properties.

8. The method, as claimed in claim 6, wherein the specified reference values of the optical properties correspond to reference values of diffuse reflectance, and
wherein the reference values of diffuse reflectance is provided as input to the DFCNN, the 1D-CNN, the merging neural network and the output neural network, during the training.

9. A device configured to predict optical properties of a sample, the device comprising:
at least one processor configured to:
obtain a plurality of diffuse reflectance values based on optical energy diffusely reflected from within the sample;
generate, by a multi-layered Deep Fully Connected Neural Network (DFCNN) in the device, a first set of intermediate values by non-linearly mapping the plurality of diffuse reflectance values to the first set of intermediate values;
generate, by a One-Dimensional-Convolutional Neural Network (1D-CNN) in the device, a second set of intermediate values by non-linearly mapping the plurality of diffuse reflectance values to the second set of intermediate values; and
predict values of the optical properties of the sample based on the first set of intermediate values and the second set of intermediate values.

10. The device as claimed in claim 9, wherein the plurality of diffuse reflectance values is provided as an input feature vector to the DFCNN, and
wherein the plurality of diffuse reflectance values correspond to features of the input feature vector.

11. The device as claimed in claim 9, wherein the plurality of diffuse reflectance values is provided as an input tensor to the 1D-CNN, and
wherein the 1D-CNN obtains shape characteristics of the plurality of diffuse reflectance values.

12. The device as claimed in claim 9, wherein the at least one processor is further configured to predict the optical properties by:
generating a merged set of intermediate layer output values by merging the first set of intermediate values and the second set of intermediate values by a merging neural network; and
reducing the merged set of intermediate values to a predefined number of output values,
wherein the intermediate values in the merged set is non-linearly mapped to a predefined number of output values by an output neural network comprising of at least one layer.

13. The device as claimed in claim 12, wherein the DFCNN, the 1D-CNN, the merging neural network, and the output neural network, are configured to be trained to predict the values of the optical properties based on a mean square weighted error cost function.

14. The device as claimed in claim 13, wherein a value of the mean square weighted error cost function is determined based on an error vector and a weight vector,
wherein the error vector is a difference between a first vector, corresponding to the values of the optical properties predicted during the training, and a second vector, corresponding to specified reference values of the optical properties, and
wherein the weight vector corresponds to weight factors assigned to the optical properties.

15. The device as claimed in claim 14, wherein magnitudes of dimensions of the weight vector is inversely proportional to ranges of the specified reference values of the optical properties, and
wherein the ranges of the specified reference values of the optical properties correspond to differences between maximum specified reference values of the optical properties and minimum specified reference values of the optical properties.

16. The device as claimed in claim 14, wherein the specified reference values of the optical properties correspond with reference values of diffuse reflectance, and
wherein the reference values of diffuse reflectance is provided as input to the DFCNN, the 1D-CNN, the merging neural network and the output neural network, during the training.
